(19) United States
(12) Patent Application Publication
(10) Pub. No.: US 2014/0171949 A1
(43) Pub. Date: Jun. 19, 2014

(54) LINEAR SLIDE INDICATOR

(71) Applicant: MATTHEW J. ATTAR, SEEKONK, MA (US)

(72) Inventor: MATTHEW J. ATTAR, SEEKONK, MA (US)

(73) Assignee: SYMMETRY MEDICAL MANUFACTURING, INC, IN (US)

(21) Appl. No.: 14/090,082

(22) Filed: Nov. 26, 2013

Related U.S. Application Data
(60) Provisional application No. 61/731,161, filed on Nov. 29, 2012.

(51) Int. Cl. A61B 17/16; A61B 17/00

(52) U.S. Cl. CPC A61B 17/1615; A61B 17/1624; A61B 17/00234
USPC 606/80

(57) ABSTRACT

The present invention provides a linear slide indicator used in connection with a medical device to indicate radial expansion or linear translation of a component within the device. The medical instrument includes a shaft, a handle, an end effector and a linear slide indicator. The shaft extends along a shaft axis and is configured to travel linearly along the shaft axis. The handle is configured to be attached to a proximal end of the shaft and to rotate around the shaft axis. The end effector is configured to be attached to a distal end of the shaft and to travel linearly with the shaft along the shaft axis via the rotational motion of the handle. The linear slide indicator is configured to indicate a magnified value of an actual traveled linear distance of the end effector.

### CROSS REFERENCE TO RELATED CO-PENDING APPLICATIONS

This application claims the benefit of U.S. provisional application Ser. No. 61/731,161 filed on Nov. 29, 2012 and entitled LINEAR SLIDE INDICATOR, which is commonly assigned and the contents of which are expressly incorporated herein by reference.

### FIELD OF THE INVENTION

The present invention relates to a linear slide indicator for a device and more particularly to a linear slide indicator used in connection with surgical devices to indicate radial expansion or linear translation of a component within a device.

### BACKGROUND OF THE INVENTION

In several surgical procedures the position of a device or of an implant within the patient's body or of a component within a device is critical to the success of the procedure. In particular, in minimally invasive surgery (MIS) instruments and implants are inserted through a cannula into an opening formed in the patient's body under fluoroscopic guidance. In these cases, linear translation or rotational motion are used to advance a component of the instrument or the implant to the appropriate surgical location within the patient's body. Linear translation or rotational motion is imparted by a moving a handle in the proximal end of an elongated shaft. In most cases, the actual distance traveled by the handle during the linear advancement of the component or the implant is small and in some cases it is almost not visible with the naked eye of the observer. Since the positioning accuracy of the device component or implant is critical, it is desirable to have a visual control and feedback mechanism indicating how far the device component or implant has advanced within the patient's body.

### SUMMARY OF THE INVENTION

The present invention provides a linear slide indicator used in connection with surgical devices to indicate radial expansion or linear translation of a component within a device.

In general, in one aspect, the invention features a medical instrument including a shaft, a handle, an end effector and a linear slide indicator. The shaft extends along a shaft axis and is configured to travel linearly along the shaft axis. The handle is configured to be attached to a proximal end of the shaft and to rotate around the shaft axis. The end effector is configured to be attached to a distal end of the shaft and to travel linearly with the shaft along the shaft axis via the rotational motion of the handle. The linear slide indicator is configured to indicate a magnified value of an actual traveled linear distance of the end effector.

Implementations of this aspect of the invention may include one or more of the following features. The medical instrument further includes a slide and a proximal shaft extending along and being collinear with the shaft axis and the slide includes outer threads and a central through-opening. The proximal shaft passes through the central through opening of the slide and the slide slides over the proximal shaft. The outer threads of the slide are configured to engage inner threads formed in an inner surface of the handle and thereby to translate the rotational motion of the handle into linear travel of the slide along the proximal shaft. The distal end of the proximal shaft is configured to connect to the shaft via a first bushing. The first bushing has a central through-opening and the central through-opening includes inner threads dimensioned to engage outer threads at the distal end of the proximal shaft and thereby to provide a small linear travel path of the first bushing that corresponds to the actual traveled linear distance of the end effector. The distal end of the proximal shaft further includes a central opening dimensioned to receive the proximal end of the shaft and to connect to the proximal end of the shaft. The outer diameter of the slide is larger than the diameter of the central through-opening of the first bushing and the outer threads of the slide have a pitch shaped and dimensioned to translate the small linear travel path of the first bushing into a magnified value of the actual traveled linear distance of the end effector. The linear slide indicator includes a linear scale indicating the magnified value of the actual traveled linear distance of the end effector. The medical instrument further includes an outer tube configured to connect to the shaft via a second bushing. The handle further includes first and second grooves shaped and dimensioned to receive the first and second bushings, respectively.

In general, in another aspect, the invention features a method for indicating a magnified value of an actual traveled linear distance of an end effector including the following. First, providing a medical instrument, that includes a shaft extending along a shaft axis and being configured to travel linearly along the shaft axis, a handle configured to be attached to a proximal end of the shaft and to rotate around the shaft axis. Next, attaching an end effector to a distal end of the shaft. The end effector is configured to travel linearly with the shaft along the shaft axis via the rotational motion of the handle. Next, providing a linear slide indicator configured to indicate a magnified value of an actual traveled linear distance of the end effector.

Among the advantages of this invention may be one or more of the following. The linear slide indicator of this invention magnifies and displays the distance between two moving components within the device.

The details of one or more embodiments of the invention are set forth in the accompanying drawings and description bellow. Other features, objects and advantages of the invention will be apparent from the following description of the preferred embodiments, the drawings and from the claims.

### BRIEF DESCRIPTION OF THE DRAWINGS

Referring to the figures, wherein like numerals represent like parts throughout the several views:

FIG. 1A depicts a perspective view of a drilling device featuring a linear slide indicator according to the invention;

FIG. 1B depicts the distal end of the drilling device of FIG. 1A;

FIG. 2A depicts the proximal end of the drilling device of FIG. 1A;

FIG. 2B depicts an enlarged view of the proximal end of the drilling device of FIG. 1A;

FIG. 3 depicts an exploded view of the proximal end of the drilling device of FIG. 1A

FIG. 4 depicts a cross-sectional diagram of the handle of the drilling device of FIG. 1A;

FIG. 5 depicts the linear slide indicator in the fully closed position and in the fully open position; and

FIG. 6 depicts a graph showing the actual linear translation of the drill bit in FIG. 1A and the magnified linear translation with the linear slide indicator according to the invention.

### DETAILED DESCRIPTION OF THE INVENTION

The present invention provides a linear slide indicator used in connection with surgical devices to indicate radial expansion or linear translation of a component within a device.

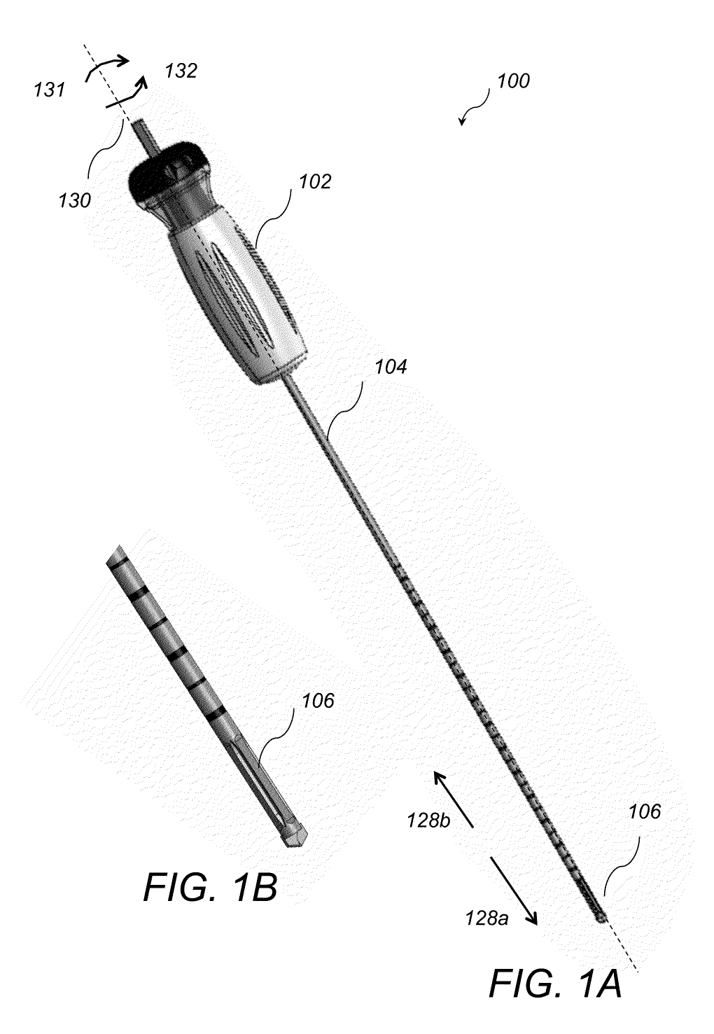

Referring to FIG. 1A and FIG. 1B, drilling instrument 100 includes an elongated tube shaft 104 having a drill bit attachment 106 at the distal end and a handle 102 at the proximal end. Clockwise rotational motion of the handle 102 along arrow 131 translates into rotational motion, expansion and linear advancement of the drill bit 106 along arrow 128a. Counter-clockwise rotational motion of the handle 102 along arrow 132 translates into rotational motion, contraction and linear advancement of the drill bit 106 along arrow 128b. The actual travel distance of the drill bit is of the order of a few millimeters and is not very visible with the naked eye.

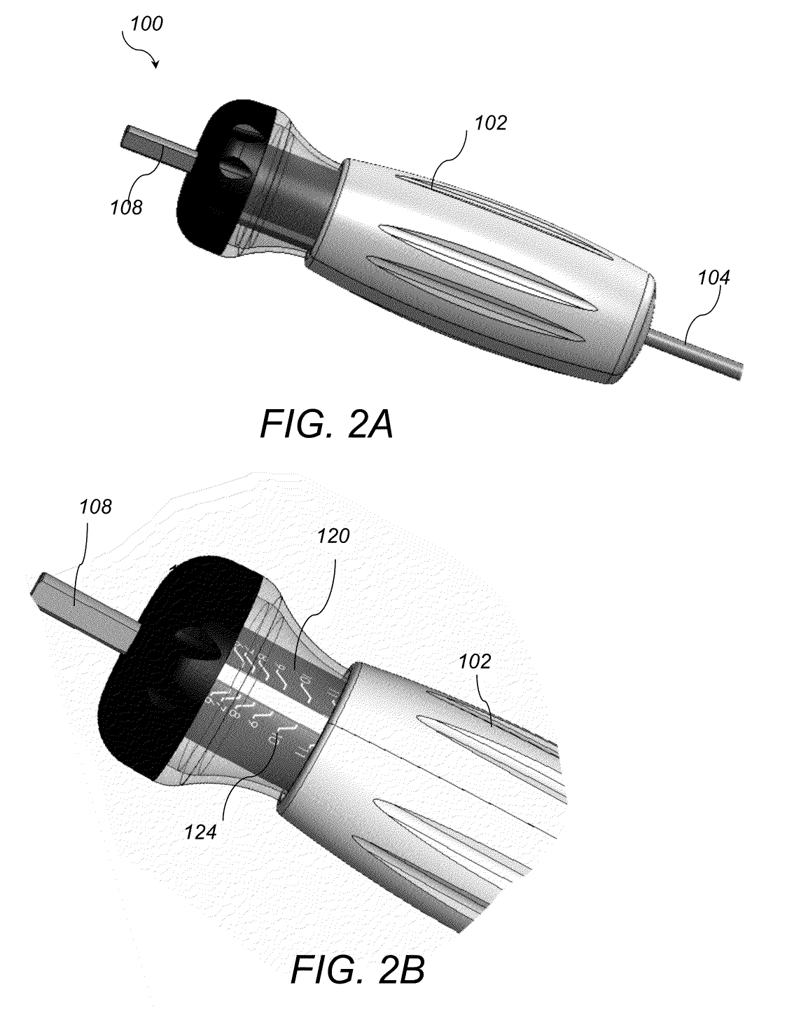

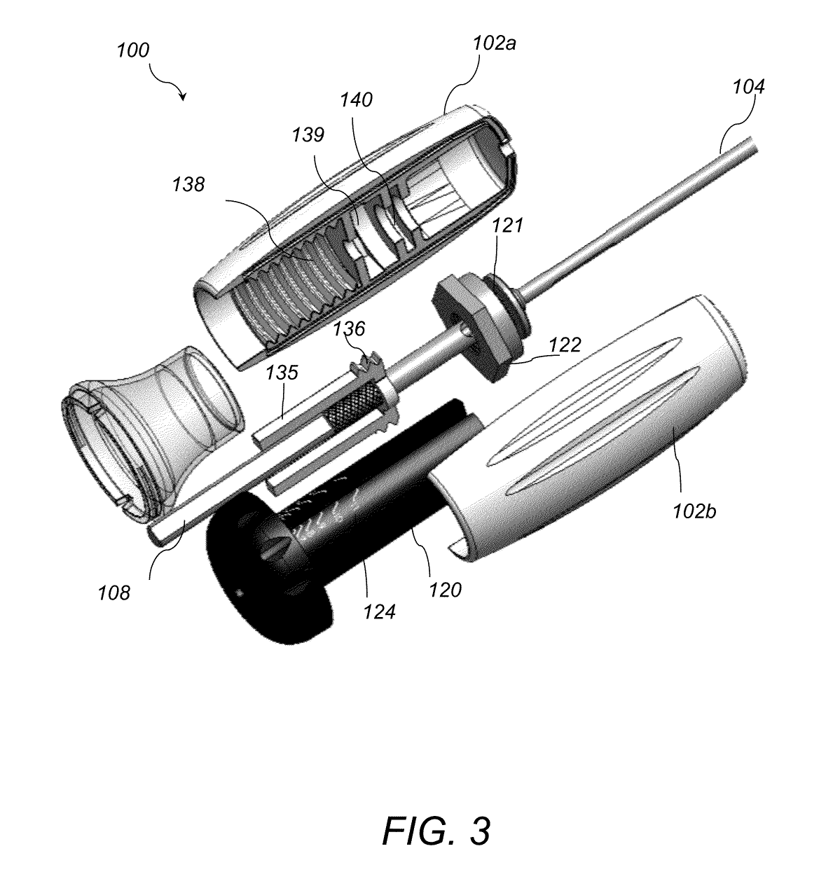

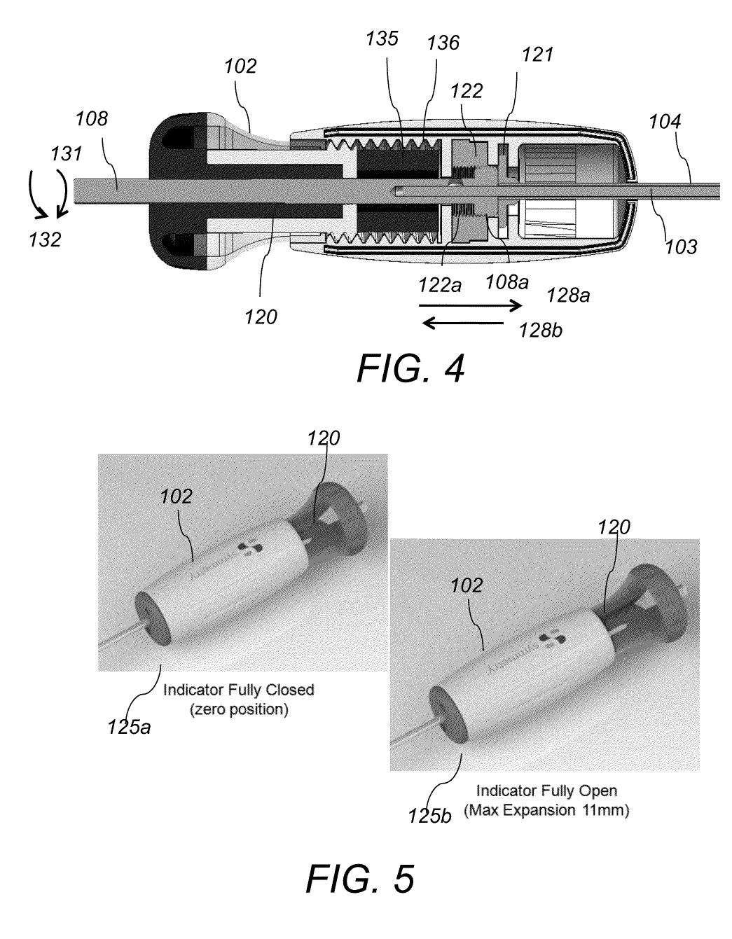

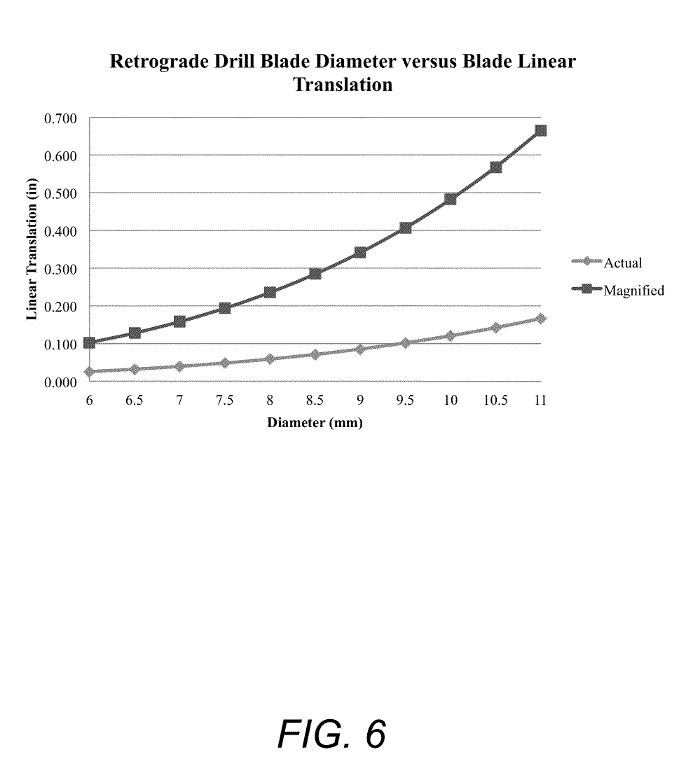

Referring to FIG. 2A, FIG. 2B, FIG. 3, and FIG. 4, the proximal end of elongated shaft 104 includes handle 102, linear slide indicator 120, slide 135, bushings 122, 121, and a proximal shaft 108. Handle 102 is configured to rotate around axis 130 clockwise 131 and counter-clockwise 132. Linear slide indicator 120 includes a scale 124 indicating a magnified value of the actual traveled distance of the drill bit. As shown in FIG. 3 and FIG. 4, within handle 102 there is shaft 108 connecting to outer tube shaft 104 via bushing 122. Outer tube shaft 104 also connects to an inner shaft 103 via bushing 121. Bushing 122 has a central through opening with inner threads 122a that are dimensioned to engage outer threads 108a at the distal end of shaft 108 and thereby to provide a small linear translation path. Handle 102 includes handle components 102a, 102b. Handle components 102a, 102b, have inner grooves 138, 139, 140 configured to house slide 135 and bushings 122, 121, respectively. Slide 135 is configured to slide over shaft 108 and has outer threads 136 that engage the inner grooves 138 of the handle 102. Slide 135 has a larger outer diameter than the diameter of the central through-opening of bushing 122 and threads 136 have a particular pitch such that the small incremental linear translation of bushing 122 is magnified to show a magnified value of the actual traveled linear distance on the larger, more readable linear scale 124. The pitch of threads 136 can be varied to accommodate different rates of translation and/or the diameter of the slide 135 can be changed in order to change the magnification and scale on the linear indicator as desired. FIG. 5 depicts a handle with the indicator 120 fully closed (125a) (zero position) and with the indicator 120 fully open (125b). In the fully open indicator position the maximum drill bit expansion is 11 mm. FIG. 6 depicts a graph showing the actual linear translation of the drill bit 106 in FIG. 1A and the magnified linear translation with the linear slide indicator 120 according to the invention. The actual and magnified linear translations of the drill bit 106 are plotted as a function of the drill bit diameter.

Several embodiments of the present invention have been described. Nevertheless, it will be understood that various modifications may be made without departing from the spirit and scope of the invention. Accordingly, other embodiments are within the scope of the following claims.

What is claimed is:

1. A medical instrument comprising:
a shaft extending along a shaft axis and being configured to travel linearly along the shaft axis;
a handle configured to be attached to a proximal end of the shaft and to rotate around the shaft axis;
an end effector configured to be attached to a distal end of the shaft and to travel linearly with the shaft along the shaft axis via the rotational motion of the handle; and
a linear slide indicator configured to indicate a magnified value of an actual traveled linear distance of the end effector.

2. The medical instrument of claim 1, further comprising a slide and a proximal shaft extending along and being collinear with the shaft axis and wherein the slide comprises outer threads and a central through-opening and wherein the proximal shaft passes through the central through opening of the slide and the slide slides over the proximal shaft.

3. The medical instrument of claim 2, wherein the outer threads of the slide are configured to engage inner threads formed in an inner surface of the handle and thereby to translate the rotational motion of the handle into linear travel of the slide along the proximal shaft.

4. The medical instrument of claim 3, wherein the distal end of the proximal shaft is configured to connect to the shaft via a first bushing.

5. The medical instrument of claim 4, wherein the first bushing comprises a central through-opening and wherein the central through-opening comprises inner threads dimensioned to engage outer threads at the distal end of the proximal shaft and thereby to provide a small linear travel path of the first bushing and wherein the small linear travel path of the first bushing corresponds to the actual traveled linear distance of the end effector.

6. The medical instrument of claim 5, wherein the distal end of the proximal shaft further comprises a central opening dimensioned to receive the proximal end of the shaft and to connect to the proximal end of the shaft.

7. The medical instrument of claim 5, wherein the outer diameter of the slide is larger than the diameter of the central through-opening of the first bushing and the outer threads of the slide comprise a pitch shaped and dimensioned to translate the small linear travel path of the first bushing into a magnified value of the actual traveled linear distance of the end effector

8. The medical instrument of claim 1, wherein the linear slide indicator comprises a linear scale indicating the magnified value of the actual traveled linear distance of the end effector.

9. The medical instrument of claim 4, further comprising an outer tube configured to connect to the shaft via a second bushing.

10. The medical instrument of claim 9, wherein the handle further comprises first and second grooves shaped and dimensioned to receive the first and second bushings, respectively.

11. A method for indicating a magnified value of an actual traveled linear distance of an end effector comprising:
providing a medical instrument, wherein the medical instrument comprises a shaft extending along a shaft axis and being configured to travel linearly along the shaft axis, a handle configured to be attached to a proximal end of the shaft and to rotate around the shaft axis;
attaching an end effector to a distal end of the shaft, wherein the end effector is configured to travel linearly with the shaft along the shaft axis via the rotational motion of the handle; and
providing a linear slide indicator configured to indicate a magnified value of an actual traveled linear distance of the end effector.

12. The method of claim 11, further comprising providing a slide and a proximal shaft extending along and being collinear with the shaft axis and wherein the slide comprises outer threads and a central through-opening and wherein the proximal shaft passes through the central through opening of the slide and the slide slides over the proximal shaft.

13. The method of claim 12, wherein the outer threads of the slide are configured to engage inner threads formed in an inner surface of the handle and thereby to translate the rotational motion of the handle into linear travel of the slide along the proximal shaft.

14. The method of claim 13, wherein the distal end of the proximal shaft is configured to connect to the shaft via a first bushing.

15. The method of claim 14, wherein the first bushing comprises a central through-opening and wherein the central through-opening comprises inner threads dimensioned to engage outer threads at the distal end of the proximal shaft and thereby to provide a small linear travel path of the first bushing and wherein the small linear travel path of the first bushing corresponds to the actual traveled linear distance of the end effector.

16. The method of claim 15, wherein the distal end of the proximal shaft further comprises a central opening dimensioned to receive the proximal end of the shaft and to connect to the proximal end of the shaft.

17. The method of claim 15, wherein the outer diameter of the slide is larger than the diameter of the central through-opening of the first bushing and the outer threads of the slide comprise a pitch shaped and dimensioned to translate the small linear travel path of the first bushing into a magnified value of the actual traveled linear distance of the end effector

18. The method of claim 11, wherein the linear slide indicator comprises a linear scale indicating the magnified value of the actual traveled linear distance of the end effector.

19. The method of claim 14, further comprising providing an outer tube configured to connect to the shaft via a second bushing.

20. The method of claim 19, wherein the handle further comprises first and second grooves shaped and dimensioned to receive the first and second bushings, respectively.
